(19) United States
(12) Patent Application Publication
(10) Pub. No.: US 2004/0147787 A1
(43) Pub. Date: Jul. 29, 2004

(54) Process for oxidation of alcohols

(76) Inventor: Ross A Miller, Fanwood, NJ (US)

Correspondence Address: MERCK AND CO INC, P O BOX 2000, RAHWAY, NJ, 070650907

(21) Appl. No.: 10/482,941

(22) PCT Filed: Oct. 16, 2001
(86) PCT No.: PCT/US01/42750

(51) Int. Cl. C07D 207/46; C07C 45/27

(52) U.S. Cl. 568/428; 548/571

(57) ABSTRACT

The use of iodine as a primary oxidant for TEMPO provides a novel and selective method for oxidizing alcohols to their corresponding carbonyl compounds.

### BACKGROUND OF THE INVENTION

The oxidation of alcohols to aldehydes and ketones is a generally, useful functional group transformation in organic chemistry. The use of N-oxoammonium salts in such oxidations has proven to be of great synthetic utility, providing products in high yields at low cost.

N-oxoammonium salts are generated with a paired system of a catalyst and a primary oxidant. The catalyst, 2,2,6,6-tetramethylpiperidine-1-oxyl (TEMPO) or a derivative thereof, is oxidized by a primary oxidant to its corresponding N-oxoammonium salt, the species that performs the oxidation of the alcohol. Many primary oxidants in the TEMPO-catalyzed oxidation have been reported, for example, bleach, N-chlorosuccinimide, acetoxyiodobenzene, m-CPBA, high-valent metal salts and electrooxidation. These primary oxidants, however, are non-chemoselective; in addition, factors such as reagent toxicity, safety, expense of reagent and waste by-product disposal render the use of these oxidants impractical for large production use.

### SUMMARY OF THE INVENTION

The present invention relates to the use of a novel primary oxidant in the TEMPO-catalyzed oxidation of alcohols to their corresponding carbonyl compounds. The process of the present invention has utility in all fields of chemical production including, but not limited to, preparation of pharmaceutical compounds and/or intermediates therefor.

### DETAILED DESCRIPTION OF THE INVENTION

The present invention provides a process for oxidizing an alcohol to its corresponding aldehyde or ketone which comprises contacting said alcohol with iodine and a catalytic amount of TEMPO or derivatives thereof in an organic solvent for a time period sufficient to effect said oxidation. In one subset, the iodine/TEMPO system is used for the oxidation of a primary alcohol to its corresponding aldehyde. In another subset, the iodine/TEMPO system is used for the oxidation of a secondary alcohol to its corresponding ketone. As used herein, unless the context indicates otherwise, TEMPO includes the substance 2,2,6,6-tetramethylpiperidine-1-oxyl as well as derivatives thereof including, but not limited to, TEMPO polystyrene, 4-hydroxy-TEMPO and 4-acetoxy-TEMPO.

The present inventors have now discovered iodine (I₂) as a novel chemoselective oxidant in the TEMPO-catalyzed oxidation; iodine is readily available, inexpensive, easy to use, and reaction by-products are easy to dispose. While the iodine/TEMPO system may be used to oxidize any alcohols to their corresponding aldehydes or ketones, it is especially suited for alcohols in which one or more aromatic rings, including heteroaromatic rings, are present in the molecule. In such molecules iodine/TEMPO selectively oxidizes the alcohol over the aromatic group or a ring nitrogen or sulfur of a heteroaromatic ring.

In the present process, iodine may be used in about 1 to about 3 equivalents relative to the alcohol substrate. TEMPO is used in catalytic amount which may be from about 0.01 to about 0.1 equivalent relative to the alcohol substrate. The reaction is carried out in an organic solvent which is non-reactive with the reagents and which does not significantly interfere with the desired reaction; examples of suitable solvents include toluene, tetrahydrofuran, acetonitrile, ethyl acetate, dichloromethane, or combinations thereof. The reaction may be carried out at a temperature range of from about 0° C. to about 100° C., and conveniently at ambient temperature of from about 20 to 25° C.

The oxidation of alcohols using iodine/TEMPO produces hydriodic acid; accordingly, for reactions in which one or more of the starting material, product, or reagents are unstable in the presence of hydriodic acid, the present process may optionally include a base to neutralize the acid thus formed. Any base that does not significantly react with the alcohol substrate, iodine, TEMPO and the carbonyl product may be used, for example, bicarbonates are suitably used. The base, if used, may be used in up to 3.5 equivalents relative to the alcohol substrate.

The iodine/TEMPO system is applicable to the oxidation of any type of primary and secondary alcohols; however, because of its chemoselectivity for the alcohol in the presence of aromatic or heteroaromatic ring, the system is especially suitable for alcohols of the formula Ar—X—CH₂OH, wherein Ar is an optionally substituted aryl or an optionally substituted heteroaryl group, and X is a bond, methylene, ethylene, or CH═CH. In one subset the alcohol is Ar—CH₂OH. “Aryl” means a 6-14 membered carbocyclic aromatic ring system comprising 1-3 benzene rings such as phenyl and naphthyl. “Heteroaryl” represents a 5-10 membered aromatic ring system containing one ring or two fused rings, 1-4 heteroatoms, selected from O, S and N; examples include furanyl, imidazolyl, isooxazolyl, isothiazolyl, oxadiazolyl, oxazolyl, pyrazolyl, pyridyl, pyrrolyl, tetrazinyl, thiazolyl, thienyl, triazinyl, triazolyl, benzothienyl, quinolinyl, benzimidazolyl, pyrrolopyridine, furopyridine and thienopyridine. The substituents on the aryl and heteroaryl groups are not particularly limited and may be any that do not significantly interfere with the oxidation of the hydroxy group.

The following examples are provided to illustrate the invention and are not intended to limit the scope of the invention in any manner. It will be understood that specific reaction conditions such as solvents, reaction temperature, amounts of reagents, and reaction times will vary with the alcohol to be oxidized, and that a person of ordinary skill in organic synthesis will be able to select the appropriate conditions without undue experimentation.

### EXAMPLE 1

### Preparation of 5-methoxy-3-benzothiophenecarboxaldehyde

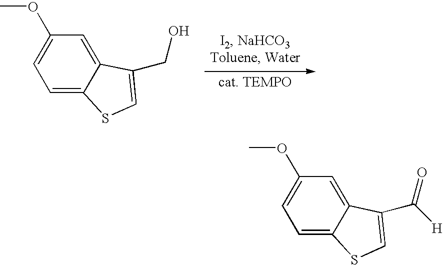

To 5-methoxy-3-benzothiophenemethanol (2.2 kg, 11.34 mole) in toluene/tetrahydrofuran was added NaHCO₃ (3.0 kg, 35.6 mole) and water (30L) and aged until the bicarbonate was dissolved. Iodine (6 kg, 23.7 mole) was added and the reaction mixture was aged for 1 hr at room temperature. TEMPO (2,2,6,6-tetramethyl-1-piperidinyloxy, free radical, 140 g, 0.90 mole) was then added. The mixture was aged at 23° C. for about 2-6 hours until the starting material was consumed.

The reaction mixture was maintained at ambient temperature and then 10% aq. Na₂SO₃ (15L) was added to quench the excess iodine. The layers were cut and the organic layer washed with water (15L). The organic layer was washed once with 2.2 kg 10% aq carbonate and then 2 kg water.

### EXAMPLE 2

### Preparation of 2-(n-butyl)-5-chloro4-imidazolecarboxaldehyde (Intermediate for Losaftan)

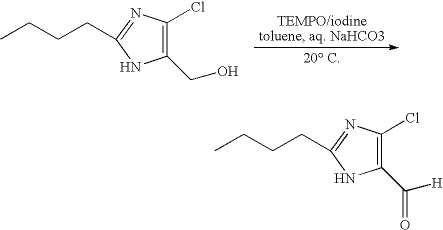

2-(n-Butyl)-5-chloro4-irdazolemethanol (750 mg, 3.98 mmol) was charged into a 100 mL round bottom flask equipped with a magnetic stir bar. The solid was then slurried in toluene (10 mL) at 20° C. An aqueous solution of sodium bicarbonate (1.00 gram, 11.94 mmol in 10 mL deionized water) was prepared and charged into the toluene slurry at 20° C. Solid iodine (2.02 grams, 7.96 mmol) was then charged in one portion to the alcohol followed by solid TEMPO (62 mg, 0.398 mmol). The reaction mixture was then aged overnight (16 hours) at 20° C. The batch was cooled to 5° C. and diluted with ethyl acetate (10 mL). The batch was quenched at 5° C. by adding an aqueous solution of sodium sulfite (501 mg sodium sulfite in 5 mL DI water). The quenched reaction mixture was transferred to a separatory funnel (rinsed with additional ethyl acetate, 10 mL and DI water, 10 mL) and the aqueous layer was cut away. The organic layer was then washed with 10 mL saturated aqueous potassium bicarbonate followed by 10 mL brine. The washed organic layer was then diluted to 50 mL

The organic layer was then dried over sodium sulfate and concentrated in vacuo to a volume of 10 mL. A mechanical stir bar was added and the solution was further concentrated by a stream of nitrogen to a volume of 5 mL. The batch was seeded with title aldehdye (50 mg) and the slurry aged at 20 C. for 30 minutes. The batch was then cooled to 5° C. and aged for 30 minutes. The solids were isolated by filtration and the cake washed with cold toluene (3 mL). The solids were dried in vacuo to provide aldehyde (690 mg).

### EXAMPLE 3

The following alcohols were oxidized to provide the corresponding aldehydes according to the general procedure described in Examples 1 and 2.

| Alcohol | Conditions |
|---|---|
| 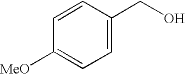 | toluene, 80° C., 30 min. |
| 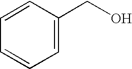 | toluene, 50-70° C., 30 min |
| 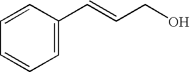 | Room temperature, 1 eq. iodine (with 2 eq. no desired product obtained). |
| 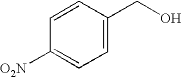 | 50-80° C. |
| 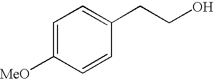 | Room temperature −50° C. in toluene |
| 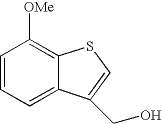 | |

What is claimed is:

1. A process for oxidizing a primary alcohol to its corresponding aldehyde which comprises contacting said primary alcohol with iodine and a catalytic amount of TEMPO or a derivative thereof in an organic solvent for a time period sufficient to effect said oxidation.

2. A process of claim 1 wherein said oxidation is carried out in the presence of a base.

3. A process of claim 1 wherein said primary alcohol is an optionally substituted aryl-C₁₋₃alkanol or an optionally substituted heteroaryl-C₁₋₃alkanol.

4. A process of claim 2 wherein said primary alcohol is an optionally substituted aryl-C₁₋₃alkanol or an optionally substituted heteroaryl-C₁₋₃alkanol.

5. A process of claim 1 wherein said solvent is toluene, tetrahydrofuran, acetonitrile, ethyl acetate, dichloromethane or a combination thereof.

6. A process for oxidizing a primary alcohol having the formula Ar—X—CH₂OH to the corresponding aldehyde having the formula Ar—X—C(O)H, wherein Ar is an optionally substituted aryl or an optionally substituted heteroaryl group, and X is a bond, C₁₋₂alkylene, —CH═CH—, or C₁₋₂alkylene—CH═CH—, which comprises contacting said primary alcohol with iodine and a catalytic amount of TEMPO or a derivative thereof in an organic solvent for a time period sufficient to effect said oxidation.

7. A process of claim 6 wherein said oxidation is carried out in the presence of a base.

8. A process of claim 6 wherein X is a bond.

9. A process of claim 7 wherein X is a bond.

10. A process for oxidizing a secondary alcohol to the corresponding ketone which comprises contacting said secondary alcohol with iodine and a catalytic amount of TEMPO or a derivative thereof in an organic solvent for a time period sufficient to effect said oxidation.

11. A process of claim 10 wherein said oxidation is carried out in the presence of a base.

12. A process for the preparation of 2-(n-butyl)-5-chloro-4-imidazolecarboxaldehyde comprising:
contacting 2-(n-butyl)-5-chloro-4-imidazolemethanol with iodine and a catalytic amount of TEMPO or a derivative thereof in the presence of a base, and in an organic solvent for a time period sufficient to effect said oxidation.

13. A process of claim 12 wherein said base is sodium bicarbonate.
